(19) United States
(12) Patent Application Publication
(10) Pub. No.: US 2008/0193489 A1
(43) Pub. Date: Aug. 14, 2008

(54) Personal Lubricant Compositions That Are Free Of Glycerin and Parabens

(76) Inventors: Robert De Armond, Temecula, CA (US); Michael B. Halsdorff, San Marcos, CA (US); Daniel X. Wray, Vista, CA (US)

Correspondence Address: Bell, Boyd & Lloyd LLP, 3580 Carmel Mountain Road, Suite 200, San Diego, CA, 92130, US

(21) Appl. No.: 11/674,617

(22) Filed: Feb. 13, 2007

(51) Int. Cl. A61K 31/70; A61P 17/00

(52) U.S. Cl. 424/400

(57) ABSTRACT

A stable personal lubricant composition comprising xylitol and excluding glycerin and parabens is provided. In one embodiment, the composition comprises from about 1% to about 15% by weight xylitol; from about 10% to about 35% by weight butylene glycol; from about 5% to about 20% by weight propylene glycol;

from about 0.1% to about 1.0% by weight polyquaternium; and from about 50% to about 70% by weight water. Also provided are methods of making and using the personal lubricant compositions comprising xylitol. Packaged kits of the compositions and articles of manufacture including the personal lubricant compositions comprising xylitol also are provided.

### FIELD OF THE INVENTION

This invention relates to personal lubricant compositions. Provided herein are compositions that are free of glycerin and parabens. The compositions of this invention provide moisture and lubricity and may be a vehicle for delivering medicaments, such as for vasodilatation and/or contraception, and for the treatment and prevention of disease to the skin and mucous membrane. This invention also relates to methods of making and using the personal lubricant compositions of this invention.

### BACKGROUND OF THE INVENTION

In recent studies, sex has been rated as the activity that produces the single largest amount of happiness (Blanchflower et al., “Money, Sex and Happiness: An Empirical Study,” National Bureau of Economic Research Working Paper Series, Working Paper 10499, JEL No. I1, J3 (2004)). In the same paper, both males and females derive happiness from sex, and the frequency of sex strongly influences the degree of happiness—“the more sex, the happier the person.”

For some women, however, intercourse may not be as pleasurable, especially with increasing age. With increasing age comes increased stress, which can decrease production of vaginal lubrication. Childbirth also can impact on vaginal lubricity. Changes in the vaginal lubrication is a leading cause of loss of pleasure during intercourse, and is the major cause of painful intercourse. When there is a lack of moisture in the vaginal tissues, the vaginal tissues become dry and irritated, resulting in sexual intercourse that can be very painful Decreased estrogen levels during menopause decreases the production of vaginal lubrication. In addition, several medications have been shown to negatively impact vaginal lubrication production, even in the fully aroused woman. These medications include antihistamines, blood pressure and heart medications, certain antidepressants and some contraceptives. Other medical conditions and factors also can contribute to decreased vaginal lubrication, including dyspareunia, vaginal atrophy, stress, and lack of sleep.

Use of personal lubricants, or sexual lubricants, can supplement the natural vaginal lubrication, greatly enhancing the sexual intercourse experience. Personal lubricant can be applied to the vagina or penis or both to ease penetration and provide the lubricity of the missing natural vaginal lubrication. There are many personal lubricants available, including those that are flavored or unflavored, thickened into a gel, applied as a liquid, foam or spray, those that provide warming or cooling and combinations thereof. The use of a personal lubricant can add to the intimacy and sensuality of the sexual experience.

Many of the commercially available personal lubricants include glycerin as a humectant to provide moisture to the mucosal surfaces to which they are applied. A very small percentage of the population reports having some sensitivity to glycerin. Thus, a need exists for a personal lubricant that is free of glycerin.

Similarly, many of the commercially available personal lubricants include a paraben as a preservative. Parabens are ubiquitous preservatives used in cosmetic formulations. Parabens have a very good safety profile. The Cosmetic Ingredient Review (CIR) reviewed the safety of methylparaben, propylparaben, and butylparaben in 1984 and concluded they were safe for use in cosmetic products at levels up to 25%. Typically parabens are used at levels ranging from 0.01 to 0.3%. In 2006, the U.S. Food and Drug Administration Center for Food Safety and Applied Nutrition stated that at the present time there is no reason for consumers to be concerned about the use of cosmetics that include parabens.

In individuals with normal skin, parabens are, for the most part, non-irritating and non-sensitizing. Parabens can, however, cause skin irritation and contact dermatitis in individuals with paraben allergies. A small percentage of the population, estimated to be less than 2%, however, have a demonstrated allergy to parabens (e.g., see Nagel et al, “Paraben Allergy,” JAMA 237(15): 1594-1595 (1977)). Accordingly, there is a need for personal lubricants that provide lubricity and moisture but that exclude parabens as ingredients in the formulation.

### SUMMARY OF THE INVENTION

The compositions and methods provided herein satisfy these as well as other needs.

It has been discovered that personal lubricants that include xylitol provide lubricity and moisture without glycerin. Compositions within the scope of this invention that include xylitol are bacteriostatic and do not require additional preservatives, such as parabens, to be shelf-stable.

A primary object of the present invention is to provide personal lubricants that impart lubricity and moisture to the skin or mucous membranes but exclude glycerin and/or parabens as ingredients.

Another object of the present invention is to provide a personal lubricant composition comprising xylitol that can be inexpensively produced or used.

The above and other objectives are accomplished by the present invention, which is directed to a personal lubricant composition comprising xylitol. In one aspect, provided herein is a topical formulation suitable for application to the skin or a mucous membrane of a subject, comprising xylitol in an amount effective to provide lubricity and moisture to the skin or mucous membrane. In another aspect, the composition comprises xylitol and butylene glycol or propylene glycol. In another aspect of the invention, the composition comprises xylitol and butylene glycol and propylene glycol. In another aspect, the composition comprises xylitol, a polyquaternium and butylene glycol or propylene glycol.

In another aspect, the composition comprises xylitol, a polyquaternium, butylene glycol and propylene glycol. In some embodiments, a surfactant or emollient is added. In some embodiments, a medication is added to the composition.

In another aspect, provided herein are methods of making and using the personal lubricant composition comprising xylitol as provided herein. In one embodiment, the personal lubricant is made by pre-hydrating xylitol in an aqueous solution and when fully hydrated, adding a humectant, such as butylene glycol or propylene glycol or a combination thereof. After thorough mixing, optional ingredients may be added and the pH may be adjusted, while constantly mixing the composition.

In another aspect of the invention, provided herein are methods of using the personal lubricant compositions comprising xylitol as provided herein. In one embodiment, provided is a method for lubricating a genital surface, comprising spreading about 0.1 mL to about 50 mL, or from about 5 mL to about 25 mL, or from about 10 mL to about 15 mL of the personal lubricant composition provided herein across one or more genital surfaces, such as surfaces inside the vagina or the surface of the penis in a manner that causes the lubricant gel to coat and remain in contact with the genital surfaces.

In another embodiment, provided herein is a method of applying a personal lubricant comprising xylitol as provided herein onto the skin of a subject, comprising dispensing about 0.1 mL to about 10 mL of the personal lubricant composition provided herein onto the skin, and rubbing the lubricant to produce a lubricating effect. In one embodiment, the personal lubricant composition is dispensed into the hand and applied to the skin, the vagina, the penis, the perianal tissue or the anus. In another embodiment, the personal lubricant is dispensed directly into the vagina or anus or onto the penis.

In another embodiment, provided herein is a method for preventing or treating vaginal dryness, comprising the step of applying the personal lubricant comprising xylitol disclosed herein inside the vagina.

In another aspect, provided herein is an article of manufacture, comprising a packaging material, within the packaging material a personal lubricant composition comprising xylitol as provided herein and formulations thereof, which is effective for providing moisture and/or lubricity to skin or a mucous membrane, and a label that indicates that the personal lubricant composition provided herein or formulation thereof, is used for applying to the skin or a mucous membrane to provide moisture and/or lubricity.

In another embodiment, the article of manufacture includes a personal lubricant composition comprising xylitol as provided herein, an applicator and instructions for application. In one embodiment, the applicator is a pump spray. In another embodiment, the applicator is adapted for delivery of a substance into a cavity in the body, such as described in U.S. Pat. Nos. D494,676, D320,084, D294,063, D279,504, D266,702, 6,537,260, 5,531,703 and 4,351,336.

In another aspect, provided herein are articles of manufacture that comprise containers in which a personal lubricant composition comprising xylitol as provided herein is sold and/or distributed. In one embodiment, these containers include a personal lubricant composition comprising xylitol as provided herein and have instructions for the self-administration of the personal lubricant. In one embodiment, the containers are metal, glass or plastic (or other appropriate inert material). In one embodiment, the instructions for use are provided on the outside of the container.

In another aspect, provided herein are articles of manufacture that include a personal lubricant composition comprising xylitol as provided herein packaged in a container equipped with a manually-operated dispensing pump mechanism, such as those known in the art. Such pump mechanisms allows a quantity of the lubricant to be conveniently dispensed when manually operated, such as by depressing a pump mechanism with one hand. In one embodiment, the package is configured to allow direct application of the personal lubricant composition directly to the vagina or penis or other mucosal surface.

In some embodiments, the personal lubricant composition is packaged as a single-use package. In one embodiment, the packets are made of plastic, metal foil, laminates or metallized plastic. In some embodiments, the packet is pre-scored or pre-notched to aid in the opening of the package. In one embodiment, the single use package comprises between about 5 mL to about 25 mL of the personal lubricant disclosed herein.

In another aspect of the invention, provided herein are kits that include a personal lubricant composition comprising xylitol as provided herein and an applicator for application of the composition. In one embodiment, the applicator is a dropper, a swab, a stick, a pump, a spray or a device adapted for delivery of a substance into a cavity of the body. In one embodiment, the applicator is a pump dispenser. In another embodiment, the kit includes a personal lubricant compositions provided herein and a prophylactic. In one embodiment, the prophylactic is a condom.

### DETAILED DESCRIPTION

A. Definitions

Unless defined otherwise, all technical and scientific terms used herein have the same meaning as is commonly understood by one of skill in the art to which claimed subject matter belongs. All patents, patent applications or published materials, including articles, books, manuals, and treatises, referred to throughout the entire disclosure herein, unless noted otherwise, are hereby expressly incorporated by reference in their entirety for any purpose. In the event that there is a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. In this application, the use of “or” means “and/or” unless stated otherwise. Furthermore, use of the term “including” as well as other forms, such as “includes,” and “included,” is not limiting.

The section headings used herein are for organizational purposes only and are not to be construed as limiting the subject matter described.

Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Reactions and purification techniques can be performed, e.g., as commonly accomplished in the art or as described herein. The foregoing techniques and procedures can be generally performed using conventional methods well known in the art and as described in various general and more specific references that are cited and discussed throughout the present specification.

As used herein, whenever a numerical range, such as 1-10 or 5% to 50%, appears herein, the range encompasses the entire range bounded by the first and last recited value. For example, “an alkyl of 1 to 20 carbon atoms” means that an alkyl group can include only 1 carbon atom, 2 carbon atoms, 3 carbon atoms, etc., up to and including 20 carbon atoms. Another example includes “a formulation including 1% to 10% by weight water,” which means that the formulation includes by weight 1%, 1.1%, 1.2%, 1.3%, 1.4%, 1.5%, 1.6%, 1.7%, 1.8%, 1.9%, 2%, 2.1% . . . , 9.7%, 9.8%, 9.9% or 10% water.

As used herein, “mammal” refers to a class of higher vertebrates comprising man and all other animals that nourish their young with milk secreted by mammary glands and that have skin that is more or less covered with hair.

As used herein, the term “subject” is an animal, typically a mammal and generally refers to a human. However, the term subject include primates, cattle, pigs, goats, sheep, cats, dogs, horses, and others in veterinary applications.

As used herein, the term “patient” includes human and animal subjects.

As used herein, “therapeutically effective amount” refers to an amount of drug sufficient to exert the desired therapeutic effect.

As used herein, “treatment” means any manner in which one or more of the symptoms of a disease or disorder are ameliorated or otherwise beneficially altered.

As used herein, “amelioration” of the symptoms of a particular disorder by administration of a particular composition refers to any lessening, whether permanent or temporary, lasting or transient that can be attributed to or associated with administration of the composition.

As used herein, “flavoring agent” refers to chemical compounds or molecules such as flavor essences or oils derived from plants, roots, beans, nuts, leaves, flowers, fruits and so forth, equivalent synthetic materials, and mixtures thereof, that are added to flavor a comestible. Flavoring agents are well known in the art.

As used herein, the term “contacting” refers to bringing two or more materials into close enough proximity that they can interact. In certain embodiments, contacting can be accomplished in a vessel such as a test tube or a petri dish. In certain embodiments, contacting can be performed in the presence of additional materials.

As used herein, the term “prophylactic” refers to a drug or device, particularly a condom, for preventing pregnancy or the transmission of a sexually transmitted disease.

As used herein, the term “condom” refers to an open-ended sheath, generally made of latex, which is worn on the penis during sexual intercourse to prevent the exchange of semen and/or possible transmission of sexually transmitted disease. Condoms are generally described in the art, such as in U.S. Pat. Nos. 6,929,118, 5,284,159, 5,010,871, and 4,869,269. The term also includes the female condom, such as is known in the art (e.g., see U.S. Pat. No. 5,325,871).

As used herein, the term “prophylactic sponge” refers to a medical device including a chemical for administration into the vaginal canal, for preventing pregnancy and/or preventing the spread of sexually transmitted diseases. Such devices are known in the art, such as described in U.S. Pat. Nos. 6,328,991 and 4,393,871.

As used herein, the term “xylitol” refers to a five-carbon sugar alcohol with the chemical formula C₅H₁₂O₅ (CAS Number 87-99-0). It is also known as (2S,3R,4R)-pentane-1,2,3,4,5-pentaol or 1,2,3,4,5-pentahydroxypentane. Xylitol is described in the Merck Index (Merck Index, 12th Edition, entry 10218 (1996)). It has the common names birch sugar and wood sugar, and occurs naturally in many vegetables and fruits. It is a low calorie sweetener, being about as sweet as sucrose or table sugar, but providing one-third fewer calories than sugar. Xylitol has no unpleasant aftertaste. It is a sugar alcohol or polyol. In man, xylitol is an intermediate product that occurs during glucose metabolism and is produced naturally in the human body through the glucuronate cycle in the liver.

As used herein, the term “polyquaternium” refers to a family of permanently charged ionic polymers that usually occurs as colorless transparent viscous liquids. This family of polymers include water soluble or water miscible cationic polymers that contain a number of quaternized nitrogen atoms. Examples of synthetic polyquaternium compounds that can be used in the present invention include, but are not limited to, polyquaternium-1, polyquaternium-2, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-22, polyquaternium-24, polyquaternium-27, polyquaternium-28, polyquaternium-29, polyquaternium-30, and mixtures thereof, wherein the compound designation is the name adopted for the compound by the Cosmetic, Toiletry and Fragrance Association, and found in the CTFA International Cosmetic Ingredient Dictionary, J. Nikitakis, ed., Cosmetic, Toiletry and Fragrance Association, Inc., Washington, D.C. (1991) and/or the CTFA Cosmetic Ingredient Handbook, John A. Wenninger and G. N. McEwen Jr., ed, Cosmetic, Toiletry and Fragrance Association, Inc., Washington, D.C. (1992). Additional examples of polyquaternium compounds which can be used in the present invention include polyquaternium-31, polyquaternium-32, polyquaternium-33, polyquaternium-34, polyquaternium-35, polyquaternium-36, polyquaternium-37, polyquaternium-39, polyquaternium-42, polyquaternium-43, polyquaternium-44, polyquaternium-45, polyquaternium-46 and polyquaternium-47.

Polyquaternium-1 has the CAS Registry Number 75345-27-6 and the chemical name Poly[(dimethyliminio)-2-butene-1,4-diyl chloride], alpha. -[4-[tris(2-hydroxy-ethyl)ammonio]-2-butenyl]-ω-[tris(2-hydroxyethyl) ammonio]-, dichloride (9Cl). Polyquaternium-2 has the CAS Registry Number 63451-27-4 and the chemical name poly[oxy-1,2-ethanediyl(dimethyliminio)-1,3-propanediyliminocarbonylimino-1,3-propanediyl(dimethyliminio)-1,2-ethanediyl dichloride] (9Cl). Polyquaternium-4 has the CAS Registry Number 92183-41-0 and the chemical name cellulose-2-hydroxyethyl ether polymer with N,N-dimethyl-N-2-propenyl-2-propen-1-ammonium chloride (9Cl). Polyquaternium-5 has the CAS Registry Number 26006-22-4 and the chemical name ethanaminium-N,N,N-trimethyl-2-[(2-methyl-1-oxo-2-propenyl)oxy]-, methyl sulfate polymer with 2-propenamide (9Cl). Polyquaternium-6 has the CAS Registry Number 26062-79-3 and the chemical name 2-propen-1-aminium, N,N-dimethyl-N-2-propenyl-chloride homopolymer (9Cl). Polyquaternium-7 has the CAS Registry Number 26590-05-6 and the chemical name 2-propen-1-aminium-N,N-dimethyl-N-2-propenyl-chloride polymer with 2-propenamide (9Cl). Polyquaternium-8 has the CAS Registry Number 130291-58-6 and the chemical name 2-propenoic acid, 2-methyl-2-(dimethylamino)ethyl ester homopolymer with bromomethane (9Cl). Polyquaternium-9 has the CAS Registry Number 130291-58-6 and the chemical name 2-propenoic acid, 2-methyl-, 2-(dimethylamino)ethyl ester homopolymer with bromomethane (9Cl). Polyquaternium-10 has the CAS Registry Number 81859-24-7 and the chemical name cellulose-2-hydroxyethyl-2-[2-hydroxy-3-(trimethylammonio)-propoxy]ethyl 2-hydroxy-3-(trimethylammonio)propyl ether chloride (9Cl). Polyquaternium-11 has the CAS Registry Number 53633-54-8 and the chemical name 2-propenoic acid, 2-methyl-, 2-(dimethylamino)-ethyl ester polymer with 1-ethenyl-2-pyrrolidinone with diethyl sulfate (9Cl). Polyquaternium-12 has the CAS Registry Number 68877-50-9 and the chemical name 2-propenoic acid, 2-methyl-, [(1R,4aR,4bR,10aR)-1,2,3,4,4a,4b,5,6,10,10a-decahydro-1,4a-dimethyl-7-(1-methylethyl)-1-phenanthrenyl]-methyl ester, polymer with 2-(diethylamino)-ethyl 2-methyl-2-propenoate and ethyl 2-methyl-2-propenoate with dimethyl sulfate (9Cl). Polyquaternium-13 has the CAS Registry Number 68877-47-4 and the chemical name 2-propenoic acid, 2-methyl-, 2-(diethylamino)-ethyl ester polymer with ethyl 2-methyl-2-propenoate and (9Z)-9-octadecenyl 2-methyl-2-propenoate with dimethyl sulfate (9Cl). Polyquaternium-14 has the CAS Registry Number 27103-90-8 and the chemical name ethanaminium-N,N,N-trimethyl-2-[(2-methyl-1-oxo-2-propenyl)oxy]-methyl sulfate homopolymer (9Cl). Polyquaternium-15 has the CAS Registry Number 35429-19-7 and the chemical name ethanaminium-N,N,N-trimethyl-2-[(2-methyl-1-oxo-2-propenyl)oxy]chloride polymer with 2-propenamide (9Cl). Polyquaternium-16 has the CAS Registry Number 95144-24-4 and the chemical name 1H-imidazolium-1-ethenyl-3-methylchloride polymer with 1-ethenyl-2-pyrrolidinone (9Cl). Polyquaternium-17 has the CAS Registry Number 148506-50-7 and the chemical name poly[oxy-1,2-ethanediyl-(dimethyliminio)-1,3-propanediylimino(1,6-dioxo-1,6-hexanediyl)imino-1,3-propanediyl-(dimethyliminio)-1,2-ethanediyl dichloride] (9Cl). Polyquaternium-18 has the CAS Registry Number 113784-58-0 and the chemical name poly[oxy-1,2-ethanediyl(dimethyliminio)-1,3-propanediylimino(1,9-dioxo-1,9-nonanediyl)imino-1,3-propanediyl(dimethyliminio)-1,2-ethanediyl dichloride] (9Cl). Polyquaternium-19 has the CAS Registry Number 110736-85-1. Polyquaternium-20 has the CAS Registry Number 110736-86-2. Polyquaternium-22 has the CAS Registry Number 53694-17-0 and the chemical name 2-propen-1-aminium-N,N-dimethyl-N-2-propenylchloride polymer with 2-propenoic acid (9Cl). Polyquaternium-24 has the CAS Registry Number 98616-25-2 and the chemical name cellulose ether with α-[3-(dodecyl-dimethylammonio)-2-hydroxypropyl]-ω-hydroxypoly(oxy-1,2-ethanediyl) chloride (9Cl). Polyquaternium-27 has the CAS Registry Number 132977-85-6 and the chemical name hexanediamide, N,N′-bis[3-(dimethylamino)propyl]-polymer with N,N′-bis[3-(dimethylamino)propyl]urea and 1,1′-oxybis[2-chloroethane], block (9Cl). Polyquaternium-28 has the CAS Registry Number 131954-48-8 and the chemical name 1-propanaminium-N,N,N-trimethyl-3-[(2-methyl-1-oxo-2-propenyl)amino]-chloride polymer with 1-ethenyl-2-pyrrolidinone (9Cl). Polyquaternium-29 has the CAS Registry Number 148880-30-2. Polyquaternium-30 has the CAS Registry Number 147398-77-4 and the chemical name ethanaminium-N-(carboxymethyl)-N,N-dimethyl-2-[(2-methyl-1-oxo-2-propenyl)oxy]-polymer with methyl 2-methyl-2-propenoate (9Cl). Polyquaternium-31 has the CAS Registry Number 189767-67-7. Polyquaternium-33 has the CAS Registry Number 69418-26-4 and the chemical name ethanaminium-N,N,N-trimethyl-2-[(1-oxo-2-propenyl)oxy]-chloride polymer with 2-propenamide (9Cl). Polyquaternium-34 has the CAS Registry Number 189767-68-8. Polyquaternium-35 has the CAS Registry Number 189767-69-9. Polyquaternium-36 has the CAS Registry Number 60494-40-8 and the chemical name 2-propenoic acid-2-methyl-2-(dimethylamino)ethyl ester polymer with methyl-2-methyl-2-propenoate with dimethyl sulfate (9Cl). Polyquaternium-37 has the CAS Registry Number 26161-33-1 and the chemical name ethanaminium-N,N,N-trimethyl-2-[(2-methyl-1-oxo-2-propenyl)oxy]chloride homopolymer (9Cl). Polyquaternium-39 has the CAS Registry Number 25136-75-8 and the chemical name 2-propen-1-aminium-N,N-dimethyl-N-2-propenylchloride polymer with 2-propenamide and 2-propenoic acid (9Cl). Polyquaternium-42 has the CAS Registry Number 31512-74-0 and the chemical name poly[oxy-1,2-ethanediyl(dimethyliminio)-1,2-ethanediyl(dimethyliminio)-1,2-ethanediyl dichloride] (9Cl). Polyquaternium-44 has the CAS Registry Number 150599-70-5 and the chemical name 1H-imidazolium-1-ethenyl-3-methyl-, methyl sulfate polymer with 1-ethenyl-2-pyrrolidinone (9Cl). Polyquaternium-46 has the CAS Registry Number 174761-16-1 and the chemical name 1H-imidazolium-1-ethenyl-3-methyl-methyl sulfate polymer with 1-ethenylhexahydro-2H-azepin-2-one and 1-ethenyl-2-pyrrolidinone (9Cl). Polyquaternium-47 has the CAS Registry Number 197969-51-0 and the chemical name 1-propanaminium-N,N,N-trimethyl-3-[(2-methyl-1-oxo-2-propenyl)amino]chloride polymer with methyl 2-propenoate and 2-propenoic acid (9Cl). The abstract associated with each of the aforementioned CAS Registry Numbers is incorporated herein by reference.

The International Cosmetic Ingredient Dictionary and Handbook (7th Edition (1997), Vol. 2, eds. J. A. Wenninger and G. N. McEwen, publ. The Cosmetic, Toiletry, and Fragrance Association, Wash. D.C.) discloses, without limitation, polyquaterniums (and their commercial sources) that are useful in the compositions of the methods of the invention. Polyquaternium-2 is commercially available from Ethox Chemicals, Inc. located in Greenville, S.C. Polyquaternium-2, polyquaternium-6, polyquaternium-7 and polyquaternium-27 are commercially available from Rhodia, Inc. located in Cranbury, N.J. Polyquaternium-4 and polyquaternium-10 are commercially available from National Starch & Chemical Company, located in Bridgewater, N.J. Polyquaternium-5, polyquaternium-6, polyquaternium-7, polyquaternium-22, polyquaternium-39 and polyquaternium-47 are commercially available from Calgon Corporation, located in Pittsburgh, Pa. Polyquaternium-6, polyquaternium-7 and polyquaternium-11 are commercially available from Tri-K Industries in Northvale, N.J. Polyquaternium-10 and polyquaternium-24 are commercially available from Amerchol Corporation located in Edison, N.J. Polyquaternium-11, polyquaternium-16, polyquaternium-44 and polyquaternium-46 are commercially available from BASF Corporation located in Washington, N.J. Polyquaternium-11 and polyquaternium-28 are commercially available from International Specially Products, located in Wayne, N.J. Polyquaternium-15, polyquaternium-35, polyquarternium-36, and polyquaternium-45 are commercially available from Rohm GmbH Chemische Fabrik located in Darmstadt, Germany. Polyquaternium-30 and polyquaternium-34 are commercially available from Chimex, located in Gonesse, France. Polyquaternium-31 is commercially available from Lipo Chemicals, Inc., located in Paterson, N.J. Polyquaternium-35 and polyquaternium-36 are commercially available from Rohm Tech, Inc. located in Maiden, Mass. Polyquaternium-42 is commercially available from Buckman Laboratories International, Inc. located in Memphis, Tenn. Polyquaternium-43 is commercially available from Clariant (France) S.A. located in Cedex, France.

As used herein, “butylene glycol” refers to an organic molecule with two alcohol groups, used in cosmetics as a humectant to bind moisture and hold water to the skin and as a solubilizer. Butylene glycol has the CAS Registry Number 107-88-0 and is also known as 1,3-butanediol and 1,3-butylene glycol. Butylene glycol is described in the Merck Index (Merck Index, 12th Edition, entry 1601 (1996)). The FDA approved butylene glycol as a direct food additive as a flavor solubilizer. It has also been granted several indirect additive approvals (e.g., see Federal Register, Vol. 62, No. 92, May 13, 1997 Part 172).

As used herein, “propylene glycol” refers to an organic molecule with two alcohol groups, used in cosmetics as a humectant and solubilizer. Propylene glycol is also known by the systematic name 1,2-propanediol. Propylene glycol has the CAS Registry Number 57-55-6, and is described in the Merck Index (Merck Index, 12th Edition, entry 8040 (1996)). It is a colorless, tasteless, hygroscopic, viscous oily liquid commonly used in personal lubricants. The Food and Drug Administration (FDA) of the United States has determined propylene glycol to be “Generally Recognized As Safe” for use in cosmetics, medicines and food. The Final Report of the CIR Expert Panel for Propylene Glycol as published in the Journal of American Toxicology (13(6): 437-491, 1994) concludes that propylene glycol is safe for use in cosmetic products at concentrations up to 50 percent.

As used herein, a composition refers to any mixture of two or more ingredients. It may be a solution, a suspension, a liquid, a powder, a paste, aqueous, non-aqueous or any combination thereof.

As used herein, the term “viscosity-modifying polymer” refers to a hydrocolloid or other polymer that when added to the compositions herein increases the viscosity thereof. The polymers modify the viscosity due to the hydrodynamic effect of the dissolved or hydrated polymers in the solvent and to the interactions between polymer molecules and the other ingredients in the composition. Viscosity-modifying polymers are well known in the art and include, but are not limited to, a cellulosic polymer, xanthan gum, an alginate, an acrylate, a methacrylate, methyl methacrylates, acrylate copolymers, a silicone, a ceramide and polyvinyl pyrrolidone. The viscosity-modifying polymer can be prehydrated in a solvent prior to adding to the final composition. The solvent may be selected from the group consisting of a water, lower alkyl alcohol, a ketone, a glycol and a mixture thereof.

As used herein, the term “paraben” refers to an ester of p-hydroxybenzoic acid, generally used as a preservative. For example, methylparaben (or methyl paraben) is the methyl ester of p-hydroxybenzoic acid, and is described in the Merck Index (e.g., see Merck Index, 12th Edition, entry 6182 (1996)). Methylparaben is also known as methyl-4-hydroxybenzoate and has the CAS Registry Number 99-76-3. Ethylparaben (or ethyl paraben) is the ethyl ester of p-hydroxybenzoic acid, and is described in the Merck Index (e.g., see Merck Index, 12th Edition, entry 3883 (1996)). Ethylparaben is also known as ethyl-4-hydroxybenzoate and has the CAS Registry Number 120-47-8. Propylparaben (or propyl paraben) is the propyl ester of p-hydroxybenzoic acid, and is described in the Merck Index (e.g., see Merck Index, 12th Edition, entry 8051 (1996)). Propylparaben is also known as propyl-4-hydroxybenzoate and has the CAS Registry Number 120-47-8. Butylparaben (or butyl paraben) is the butyl ester of p-hydroxybenzoic acid, and is described in the Merck Index (e.g., see Merck Index, 12th Edition, entry 1619 (1996)). Butylparaben is also known as butyl-4-hydroxybenzoate and has the CAS Registry Number 94-26-8. Isobutylparaben (or isobutyl paraben) is the isobutyl ester of p-hydroxybenzoic acid. Isobutylparaben is also known as isobutyl-4-hydroxybenzoate and has the CAS Registry Number 4247-02-3. Benzylparaben (or benzyl paraben) is the benzyl ester of p-hydroxybenzoic acid. Benzylparaben is also known as benzyl-4-hydroxybenzoate and has the CAS Registry Number 94-18-8. The term “parabens” includes one or a combination of p-hydroxybenzoic acid esters and salts thereof, including sodium and potassium salts of the benzoate ester.

As used herein, the term “ginsenosides” refers to a class triterpene saponins found exclusively in ginseng plants (plants of the genus Panax). The various ginsenosides are separated by column chromatography or produced by culturing ginseng (Panax ginseng) embryogenic tissues in bioreactors (e.g., see Asaka et al., Biotechnology Letters 15(12): 1259-1264 (1993). Researchers have identified more than 40 different ginsenosides. These include Ra1, Ra2, Ra3, Rb1, Rb2, Rb3, Rc, Rd, Re F2, Rg1, Rg2, Rg3, Rg4, Rg5, Rh1, Rh2 and Rh3 (see, e.g., Yu et al., Chem. Pharma Bull. 55(2): 231-235 (2007). Research studies suggest that it is the ginsenosides that are pharmaceutically active (e.g., see Yu et al., Chem. Pharma Bull. 55(2): 231-235 (2007), Kim et al., Biol Pharm Bull. 29(12):2472-2478 (2006) and Tsai et al, Chinese J Physiology 46(1):1-7 (2002)).

As used herein, the recitation “composition is free of” means that the composition excludes or does not include the stated ingredient. For example, “composition free of glycerin” means that the composition does not include glycerin and excludes glycerin as an ingredient. Similarly, “composition free of parabens” means that the composition does not include parabens and excludes parabens as an ingredient.

As used herein, the term terms “lubricant,” “lubricating,” and “lubrication” refer to any composition of matter that serves to reduce friction between an individual's tissue and another object. The “lubricant” provides slipperiness or lubricity when rubbed against the surface of skin and/or mucosal tissue.

As used herein, “lubricity” refers to the property or state of being lubricious, having a smooth or slippery quality or of having a reduced perception of friction.

As used herein, a “combination” refers to any association between two or more items.

As used herein, “fluid” refers to any composition that can flow. Fluids thus encompass compositions that are in the form of liquids, semi-solids, pastes, solutions, aqueous mixtures, gels, lotions, creams and other such compositions.

Unless otherwise indicated, as expressed in the present specification, % (percent) refers to % wt/wt.

### B. Compositions

The personal lubricant compositions provided herein comprise an aqueous solution of xylitol and exclude glycerin and/or preservatives. The personal lubricant compositions provided herein are bacteriostatic. In one embodiment, the personal lubricant comprises from about 1% to about 15% xylitol and is free of glycerin and/or preservatives. In one embodiment, the personal lubricant comprises from about 1% to about 15% xylitol and the composition does not include glycerin. In another embodiment, the personal lubricant comprises from about 1% to about 15% xylitol and the composition does not include a paraben. In another embodiment, the personal lubricant comprises from about 1% to about 15% xylitol and the composition does not include glycerin or a paraben. In other embodiments, the composition comprises 1%, 2%, 3%, 4%, 5%, 6%, 7%, 8%, 9%, 10%, 11%, 12%, 13%, 14% or 15% xylitol by weight of the composition and the composition does not include glycerin. In other embodiments, the composition comprises 1%, 2%, 3%, 4%, 5%, 6%, 7%, 8%, 9%, 10%, 11%, 12%, 13%, 14% or 15% xylitol by weight of the composition and the composition does not include a paraben. In other embodiments, the composition comprises 1%, 2%, 3%, 4%, 5%, 6%, 7%, 8%, 9%, 10%, 11%, 12%, 13%, 14% or 15% xylitol by weight of the composition and the composition does not include glycerin or a paraben.

In another embodiment, the composition comprises from about 1% to about 15% xylitol and butylene glycol, and is free of glycerin and/or preservatives. In another embodiment, the composition comprises from about 1% to about 15% xylitol and from about 10% to about 35% butylene glycol, and is free of glycerin and/or preservatives. In another embodiment, the composition comprises 1%, 2%, 3%, 4%, 5%, 6%, 7%, 8%, 9%, 10%, 11%, 12%, 13%, 14% or 15% xylitol by weight of the composition and 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34% or 35% butylene glycol by weight of the composition, and is free of glycerin and/or preservatives. In another embodiment, the composition comprises 1%, 2%, 3%, 4%, 5%, 6%, 7%, 8%, 9%, 10%, 11%, 12%, 13%, 14% or 15% xylitol by weight of the composition and 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34% or 35% butylene glycol by weight of the composition, and the composition does not include glycerin or a paraben.

In another embodiment, the composition comprises from about 1% to about 15% xylitol And propylene glycol, and is free of glycerin and/or preservatives. In another embodiment, the composition comprises from about 1% to about 15% xylitol and from about 5% to about 20% propylene glycol, and is free of glycerin and/or preservatives. In another embodiment, the composition comprises 1%, 2%, 3%, 4%, 5%, 6%, 7%, 8%, 9%, 10%, 11%, 12%, 13%, 14% or 15% xylitol by weight of the composition and 5%, 6%, 7%, 8%, 9%, 10%, 11%, 12%, 13%, 14%, 15%, 16%, 17%, 18%, 19% or 20% propylene glycol by weight of the composition, and is free of glycerin and/or preservatives. In another embodiment, the composition comprises 1%, 2%, 3%, 4%, 5%, 6%, 7%, 8%, 9%, 10%, 11%, 12%, 13%, 14% or 15% xylitol by weight of the composition and 5%, 6%, 7%, 8%, 9%, 10%, 11%, 12%, 13%, 14%, 15%, 16%, 17%, 18%, 19% or 20% propylene glycol by weight of the composition, and the composition does not include glycerin or a paraben.

In another embodiment, the composition comprises from about 1% to about 15% xylitol and polyquaternium, and is free of glycerin and/or preservatives. In another embodiment, the composition comprises from about 1% to about 15% xylitol and from about 0.1% to about 1.0% polyquaternium, and is free of glycerin and/or preservatives. In another embodiment, the composition comprises 1%, 2%, 3%, 4%, 5%, 6%, 7%, 8%, 9%, 10%, 11%, 12%, 13%, 14% or 15% xylitol by weight of the composition and 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95% or 1.0% polyquaternium by weight of the composition, and is free of glycerin and/or preservatives. In another embodiment, the composition comprises 1%, 2%, 3%, 4%, 5%, 6%, 7%, 8%, 9%, 10%, 11%, 12%, 13%, 14% or 15% xylitol by weight of the composition and 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95% or 1.0% polyquaternium by weight of the composition, and the composition does not include glycerin or a paraben.

In one embodiment, the polyquaternium is selected from among polyquaternium-1, polyquaternium-2, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-22, polyquaternium-24, polyquaternium-27, polyquaternium-28, polyquaternium-29, polyquaternium-30, polyquaternium-31, polyquaternium-32, polyquaternium-33, polyquaternium-34, polyquaternium-35, polyquaternium-36, polyquaternium-37, polyquaternium-39, polyquaternium-42, polyquaternium-43, polyquaternium-44, polyquaternium-45, polyquaternium-46, polyquaternium-47 and mixtures thereof. In another embodiment, the polyquaternium is polyquaternium-5, polyquaternium-7, polyquaternium-15, polyquaternium-32 or polyquaternium-33. In another embodiment, the polyquaternium is polyquaternium-15.

In another embodiment, the composition comprises from about 1% to about 15% xylitol, butylene glycol, propylene glycol and polyquaternium, and is free of glycerin and/or preservatives. In another embodiment, the composition comprises from about 1% to about 15% xylitol, from about 10% to about 35% butylene glycol, from about 5% to about 20% propylene glycol and from about 0.1% to about 1.0% polyquaternium, and is free of glycerin and/or preservatives. In another embodiment, the composition comprises from about 1% to about 15% xylitol, from about 10% to about 35% butylene glycol, from about 5% to about 20% propylene glycol and from about 0.1% to about 1.0% polyquaternium, and is free of glycerin and a paraben.

In another embodiment, the composition comprises 1%, 2%, 3%, 4%, 5%, 6%, 7%, 8%, 9%, 10%, 11%, 12%, 13%, 14% or 15% xylitol by weight of the composition, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34% or 35% butylene glycol by weight of the composition, 5%, 6%, 7%, 8%, 9%, 10%, 11%, 12%, 13%, 14%, 15%, 16%, 17%, 18%, 19% or 20% propylene glycol by weight of the composition, and 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95% or 1.0% polyquaternium by weight of the composition, and is free of glycerin and a paraben.

In one embodiment, the compositions provided herein further include an antioxidant. Exemplary antioxidants include, but are not limited to, ascorbic acid, butylated hydroxyl anisole, butylated hydroxy toluene, propyl gallate, tartaric acid, phosphoric acid, erythrobic acid, lactic acid, sodium sulfite, sodium bisulfate, sodium metabisulfite, thioglycolic acid, cysteine hydrochloride, a tocopherol or combinations thereof. In some embodiments, the antioxidant is at least one compound selected from among ascorbic acid, butylated hydroxyl anisole, butylated hydroxy toluene, propyl gallate, tartaric acid, phosphoric acid, erythrobic acid, lactic acid, sodium sulfite, sodium bisulfate, sodium metabisulfite, thioglycolic acid, cysteine, hydrochloride, and tocopherols. The antioxidant may be present in salt forms. In some embodiments, the salt form of an antioxidant includes ascorbic acid, tartaric acid, phosphoric acid, erythrobic acid, and lactic acid. In some embodiments, the antioxidant is a tocopherol. In one embodiment, the antioxidant is present in an amount of about 0.025% to about 10%. In one embodiment, the antioxidant is present in an amount of about 0.1% to about 5%.

In some embodiments, a viscosity-modifying polymer is included in the formulation. The viscosity-modifying polymer is selected from the group consisting of a cellulosic polymer, xanthan gum, guar gum, an alginate, acrylates, methacrylates, silicones, carboxypolymethylene, polyacrylamide and polyvinyl pyrrolidone. The viscosity-modifying polymer can be prehydrated in a solvent prior to adding to the final composition. The solvent is selected from the group consisting of a water, lower alkyl alcohol, a ketone, a glycol and a mixture thereof.

In another embodiment, the compositions provided herein include silicone to impart lubricity or viscosity. Suitable silicones that can be used in the compositions provided herein include, without limitation, nonvolatile silicone fluids such as dimethicone copolyol, cyclomethicone, polydimethylsiloxane, cyclic dimethyl polysiloxane, aminosilicones, phenylsilicones, diphenyldimethicones, phenyltrimethicones, cyclopentasiloxane, dimethicone copolyol (a polymer of dimethyl-siloxane with polyoxyethylene and/or polyoxypropylene), dimethicone copolyol, cetyldimethicone copolyol, cetyl dimethicone, cetyl dimethiconecopolyol, and amino-propyl PEG-7 PEG-3 dimethicone copolyol and dimethiconols. Such silicones are readily available from Goldschmidt A G, General Electric (Waterford, N.Y.), and Dow Corning Corporation (Midland, Mich.). The silicone oils are generally selected to have viscosities in the range about 20 to about 10000 mPa·s. In some embodiments, the silicone is selected from among cyclomethicone and dimethicone and silicone elastomers such as dimethicone/vinyl dimethicone crosspolymer, dimethicone crosspolymer, cyclopentasiloxane, polysilcone-11 and those described in Starch, “New Developments in Silicone Elastomers in Skin Care” (2002) and in U.S. Pat. Nos. 5,919,437, 5,919,468, 5,266,321, 5,654,362, 5,412,004 and 5,871,761, the disclosure of each of which is specifically incorporated by reference herein.

In some embodiments, the viscosity-modifying polymer includes a cellulosic polymer. In one embodiment, the cellulosic polymer is selected from among hydroxylethyl cellulose, hydroxy propyl cellulose, hydroxy propyl methyl cellulose, methyl cellulose, carboxy methyl cellulose, sodium carboxy methyl cellulose, and ethyl cellulose. In some embodiments, the thickener is a ceramide, as described in U.S. Pat. No. 5,665,699.

In one embodiment, a cellulosic polymer is included in the compositions of this invention in an amount of from about 0.1% to about 2% by weight of the composition. In another embodiment, a cellulosic polymer is present in an amount of from about 0.25% to about 1%. It should be noted that raising the cellulose polymer level may negatively impact lubricity, and the lubricity of the compositions may decrease with increasing cellulose polymer level. Increasing the cellulose polymer concentration results in an increase in the viscosity of the composition, which may increase the coefficient of friction and thereby may decrease the lubricity.

In one embodiment, the pH of the composition is adjusted to be compatible with the vaginal, rectal or oral membranes or to be compatible with the pH of the surface to which the composition will be delivered. An inorganic base may be used to adjust the pH of the composition from acidic to more neutral. Potassium hydroxide, sodium hydroxide or another alkali metal or alkaline earth metal base may be useful to provide the appropriate pH. Any other physiologically acceptable base may also be used in this manner to adjust the pH from acidic to more neutral. From about 0.05 to about 5% by weight inorganic base may be used. An organic or inorganic acid may be used to adjust the pH of the composition from alkaline to more neutral to be compatible with the vaginal, rectal or oral membranes or to be compatible with the pH of the surface to which the composition will be delivered. An exemplary inorganic acid is hydrochloric acid. Exemplary organic acids useful to adjust the pH of the composition include citric acid, adipic acid, lactic acid, boric acid, malic acid, succinic acid, tartaric acid and combinations thereof.

In one embodiment, the personal lubricant composition of this invention maintains its pH in the range that is most compatible with the vagina and other biomembranes. In one embodiment, the pH is in the range of from about 3 to about 6. In one embodiment, the pH is in the range of from about 3.5 to about 5. In one embodiment, the pH is about 4. In one embodiment, the pH is about 5.5.

In another embodiment, the compositions provided herein are formulated for topical administration and comprise a humectant and/or an emollient. In one embodiment, the humectant and/or emollient is selected from among alkylmonoglycerides, alkyldiglycerides, triglycerides, derivatives of these oils such as hydrogenated oils, diols such as 1,2-propanediol, 1,3-butanediol, cetyl alcohol, stearyl alcohol, oleic alcohol, glycol, butylene glycol, polyethylene glycol esters, polyethylene glycols or polypropylene glycols, fatty esters such as isopropyl palmitate, 2-ethylhexyl cocoate, myristyl myristate, isopropyl myristate, isopropyl stearate, glyceryl triacetate, ascorbyl palmitate, octyl dodecanol, hexyl decanol, oleyl alcohol, decyl oleate, hexyl laureate, dioctyl cyclohexane, glycearyl stearate, Ceteareth 20, Cetereath 12, cetyl palmitate, esters of lactic acid, stearic acid, behenic acid, isostearic acid, lecithin, collagen, elastin, keratin, lanolin, polydextrose, synthetic alcohols such as benzyl alcohol, butyl alcohol, cetearyl alcohol, cetyl alcohol, myristyl alcohol, diglyceryl caprylate and combinations thereof. In one embodiment, the emollient comprises from about or at 1% to about or at 20% of the composition. In another embodiment, the emollient comprises 1%, 2%, 3%, 4%, 5%, 6%, 7%, 8%, 9%, 10%, 11%, 12%, 13%, 14%, 15%, 16%, 17%, 18%, 19% or 20% of the composition.

In one embodiment, the composition optionally comprises additional constituents selected from among flavoring agents, fragrances, aromas, medicinals and colorants.

Examples of suitable flavoring agents include, but are not limited to, natural or artificial fruit flavors, such as lemon, orange, banana, grape, lime, apricot, grapefruit, apple, strawberry and cherry, chocolate, pineapple, coffee, vanilla, cocoa, cola, peanut, almond, licorice and cinnamon. The amount of flavoring agent employed is a matter of preference but in general a flavoring agent is used in amounts up to about or at 5%, usually from about or at 0.1% to about or at 1%, by weight of the composition. The flavoring agents can be used alone or in any combination. Some flavoring agents may be used as masking agents to cover or mask undesirable flavor notes or attributes.

In one embodiment, the personal lubricant compositions of this invention comprise a contraceptive or a spermicidal compound. Preferred for use in the compositions of this invention are the alkylphenoxypolyethoxyethanol surfactant spermicides such asp-nonylphenoxy-polyethoxyethanol (known as nonoxynol-9) and p-octylphenoxypolyethoxy-ethanol, menfegol and those described in U.S. Pat. No. 2,943,979. In one embodiment, a contraceptive is present at a concentration of from about 1% to about 15% contraceptive by weight of the composition.

In one embodiment, the personal lubricant compositions of this invention comprise one or more vasodilators. In one embodiment, the vasodilator is selected from the group consisting of prostaglandins, endothelial-derived relaxation factors, vasoactive intestinal polypeptide agonists, smooth muscle relaxants, leukotriene inhibitors, and combinations thereof. In one embodiment, the vasodilator is a prostaglanidin. In another embodiment, the vasodilator is a prostaglandin selected from among PGE₀, PGE₁ and PGE₂, or a combination thereof.

In one embodiment, the personal lubricant compositions of this invention further comprise one or more additional ingredients selected from among L-arginine, L-ornithine, L-aspaitic acid, ginko biloba, citrulline, deer velvet antler extract, Siberian ginseng extract, a tocopherol, a tocotrienol, a ginsenoside and pycnogenol and combinations thereof. In one embodiment, the personal lubricant composition includes a combination of L-arginine and L-ornitihine. In another embodiment, the personal lubricant composition includes a phyto-tocopherol. In another embodiment, the personal lubricant composition includes a phyto-tocotrienol.

In one embodiment, the personal lubricant compositions of this invention comprise an antifungal agent. In one embodiment, the antifungal agent is an imidazole. In another embodiment, the personal lubricant compositions comprise an antifungal agent selected from miconazole, econazole, terconazole, saperconazole, itraconazole, butaconazole, dotrimazole, tioconazole and ketoconazole. In one embodiment, the antifungal agent is present in the formulation at a concentration of from about 1 to about 10% antifungal agent by weight of the composition.

### C. Methods of Making the Compositions

The compositions of this invention were prepared using good manufacturing practices and optionally were prepared under aseptic conditions. First, a 60% solution of xylitol in water was prepared. A vessel capable of being heated was charged with 40 parts purified water and heated to 40° C.-50° C. with mixing using a Lightning® mixer and a paddle blade. When the temperature of the mixture reached 40° C.-50° C., 60 parts xylitol (which was in crystalline form) was slowly added with mixing and with heating to maintain a temperature between about 40° C.-50° C. (the solution of xylitol causes the solution to cool). Upon complete addition of the xylitol, the solution was mixed continuously until all of the xylitol was dissolved, as determined by a visual inspection of the solution, which was transparent and free of any visible xylitol crystals. The resulting solution was a clear colorless liquid with no observable particulates. To make an aqueous composition as instantly claimed that includes 6% xylitol, 10% propylene glycol, 20% butylene glycol and 0.4% polyquaternium, the purified water (59.6%) was dispensed into a container fitted with a paddle mixer, and the polyquaternium was added with mixing. The polyquaternium was allowed to hydrate with mixing for about 15 minutes. After 15 minutes, it was verified that all solid materials were dissolved.

After 15 minutes of mixing of the polyquaternium solution, the propylene glycol, butylene glycol and xylitol solution were dispensed with mixing into the vessel containing the hydrated polyquaternium. Mixing was continued for 30 minutes at a slow (200-500 rpm) speed. The resulting solution was smooth and clear with no visible particulate matter. The pH optionally is adjusted at this point, if necessary. If the pH is too acidic, a non-organic alkaline is added with mixing. If the pH is too alkaline, a non-organic or organic acid is added with mixing. Optional ingredients, such as flavoring agents, fragrances, aromas, medicinals and colorants, are added at this point with constant mixing to insure hydration and/or dispersal of the optional ingredients throughout the composition.

### D. Methods of Use of the Compositions

In one embodiment, provided herein is a method of applying a personal lubricant comprising xylitol as provided herein onto the internal and external surfaces of a latex condom, comprising contacting the latex condom with about 0.1 mL to about 10 mL of the personal lubricant composition provided herein and packaging the condom under conditions that promote the migration of the lubricant onto the condom.

In another embodiment, provided herein is a method of applying a personal lubricant comprising xylitol as provided herein onto the skin and/or mucous membrane of a subject, comprising dispensing about 0.1 mL to about 10 mL of the personal lubricant composition provided herein onto the skin and/or mucous membrane, and rubbing the lubricant to produce a lubricating effect. In one embodiment, the personal lubricant composition is dispensed into the hand and applied to the skin, the vagina, the penis, the perianal tissue, the anus or other mucous membrane. In another embodiment, the personal lubricant is dispensed directly into the vagina or anus or onto the penis.

In another embodiment, provided herein is a method for preventing or treating vaginal dryness, comprising the step of applying the personal lubricant comprising xylitol disclosed herein inside the vagina.

In another embodiment, provided is a method for lubricating a genital surface, comprising spreading about 0.1 mL to about 10 mL of the personal lubricant composition provided herein across one or more genital surfaces, such as surfaces inside the vagina or the surface of the penis in a manner that causes the lubricant gel to coat and remain in contact with the genital surfaces.

### E. Articles of Manufacture

In some embodiments, the compositions provided herein and formulations thereof are packaged as articles of manufacture containing a packaging material, within the packaging material a personal lubricant composition provided herein and formulations thereof, and a label that indicates that the personal lubricant composition provided herein or formulation thereof, is useful for providing lubrication and/or moisture.

The articles of manufacture provided herein include packaging materials. Packaging materials for use in packaging products are well known to those of skill in the art (see, e.g., U.S. Pat. Nos. 5,323,907, 5,052,558 and 5,033,252). Examples of packaging materials include but are not limited to, blister packs, bottles, tubes, vials, jars, containers, foil packets, aerosol bottles and devices, and any packaging material suitable for a selected formulation and intended mode of administration and treatment. A wide array of formulations of the compositions provided herein and formulations thereof are contemplated.

In certain embodiments, the compositions are presented in the form of a unit dosage form, such as a self-contained delivery device, such as a suppository or an encapsulated bead in a gelatin coating, such as is common in the art for distribution of bath oils (e.g., see U.S. Pat. Nos. 5,254,294 and 4,597,885) in a pack or dispenser device, which may include one or more unit dosage forms containing a composition provided herein. The pack may, for example, include metal or plastic foil, such as a blister pack. The pack or dispenser device may be accompanied by instructions for administration. Compositions provided herein also may be prepared, placed in an appropriate container, and labeled for appropriate use, such as application to the genitals.

In another embodiment, provided herein are containers in which the compositions of the subject invention are sold and/or distributed. In one embodiment, these containers include the personal lubricant formulations provided herein and have instructions for the use of the personal lubricant compositions provided herein. In another embodiment, the containers are glass, metal or plastic (or other appropriate inert material). In one embodiment, the formulation is prepared for immediate use. In one embodiment, the instructions for use are written on the outside of the container. In another embodiment, the personal lubricant formulation is packaged in a plastic bottle, tube or vial, which includes instructions for use thereof on the outside of the bottle, tube or vial, which includes an easy to open closure, such as a pump-dispenser type device as part of a cap assembly or flip-top closure, that renders it convenient and easy to use during sexual activity.

In some embodiments, the personal lubricant formulation is packaged in a watertight tube made of deformable metal or plastic that is sealed at one end and has a removable closure or cap at the other, such as is used to contain and dispense toothpaste. The cap may be a screw-on type that must be removed completely to dispense the contents, or may have a hinged flip-type cap that can be opened without detaching it from the tube. An advantage of the flip-type cap is that is can be easily opened or closed with one hand. The lubricant is dispensed by squeezing the tube.

In another embodiment, the personal lubricant formulations provided herein are packaged in a container equipped with a manually-operated dispensing pump mechanism, such as those known in the art (e.g., see U.S. Pat. Nos. 6,286,732, 6,006,949 and 5,405,057). Such pump mechanisms allows a quantity of the lubricant to be conveniently dispensed when manually operated, such as by depressing a pump mechanism with one hand. In one embodiment, the package is configured to allow direct application of the personal lubricant composition directly to the vagina or penis or other mucosal surface.

In some embodiments, the personal lubricant composition is packaged as a single-use package. In one embodiment, the packets are made of plastic, metal foil, laminates or metallized plastic. In some embodiments, the packet is pre-scored or pre-notched to aid in the opening of the package. In one embodiment, the single use package comprises between about 5 mL to about 25 mL of the personal lubricant disclosed herein.

In another embodiment, the personal lubricant composition is packaged within an applicator as a single-use package. In one embodiment, the applicator is a vaginal applicator (e.g., see U.S. Pat. Nos. D494,676, D320,084, D294,063, D279,504, D266,702) or other device adapted for delivery of a substance to a cavity in the body (e.g., see U.S. Pat. Nos. 6,537,260, 5,531,703 and 4,351,336).

### F. Kits

In another embodiment, the present invention also provides kits. In various embodiments the kits include a composition of the present invention in a package or other enclosure, instructions for use, and optionally an applicator. In another embodiment, the kit is provided in a wrapping (such as a plastic) that surrounds the kit. In one embodiment, the applicator is provided inside the package. In other embodiments, the packaging is selected from among a cardboard or paper box, a plastic pouch or a foil pouch. Packaging for the formulation is generally not critical, and there are a number of ways in which the personal lubricant composition may be packaged.

In one embodiment, the kit includes a personal lubricant composition provided herein and an applicator for application of the composition. In one embodiment, the applicator is a dropper, a swab, a stick, a pump, a spray or a syringe. In one embodiment, the applicator is a pump dispenser. In another embodiment, the kit includes a personal lubricant compositions provided herein and a prophylactic. In one embodiment, the prophylactic is a condom.

The compositions and methods provided herein will be further illustrated in the following, non-limiting examples. The Examples are illustrative of various embodiments only and do not limit the scope of the invention in anyway.

### EXAMPLE 1

### Exemplary Formulation

An exemplary composition in accordance with this invention was made including the following ingredients:

| Ingredient | % weight of composition |
|---|---|
| Propylene glycol | 10 |
| Butylene Glycol | 20 |
| Polyquaternium-15 | 0.40 |
| Xylitol solution (60%) | 10 |
| Purified water | 59.6 |
| Total = | 100 |

The compositions of this invention were prepared as follows. The compositions were prepared using good manufacturing practices. First, a 60% solution of xylitol in water was prepared. A aseptic vessel (heat sterilized or sterilized by wiping down with isopropyl alcohol) capable of being heated was charged with 40 parts purified water (400 g). The water was heated to 40° C.-50° C. with mixing using a paddle mixer. To the water, 60 parts xylitol (600 g) slowly was added with mixing and with heating to maintain a temperature between about 40° C.-50° C. Upon complete addition of the xylitol, the solution was mixed continuously until all of the xylitol was dissolved, as determined by a visual inspection of the solution. The resulting solution was a clear colorless liquid with no observable particulates.

To make the exemplary composition, the purified water was dispensed into a container fitted with a paddle mixer, and the polyquaternium-15 was added with mixing. The polyquaternium-15 was allowed to hydrate with mixing for about 15 minutes. After 15 minutes of mixing of the polyquaternium-15 solution, the propylene glycol, butylene glycol and xylitol solution were dispensed with mixing into the vessel containing the hydrated polyquaternium-15. Mixing was continued for 30 minutes.

The resulting solution was allowed to rest for 24 hours, followed by a second mixing for 30 minutes at slow speed using the paddle mixer. The resulting solution was smooth and clear with no visible particulate matter.

### EXAMPLE 2

### Antimicrobial Activity/Stability

The exemplary formulation described in Example 1 was tested for antimicrobial stability using the United States Pharmacopeia test method <51>, entitled Antimicrobial Effectiveness Testing (see <51> Antimicrobial Effectiveness Testing, United States Pharmacopeia 29:2499-2500 (2006). This test method is specifically incorporated by reference herein in its entirety. The test microorganisms used included a mold (Aspergillus niger), a yeast (Candida albicans), and three bacteria (E. coli, Pseudomonas aeruginosa and Staphylococcus aureus). The results are shown in Table 1 below. The criteria for acceptance for the antimicrobial preservative effectiveness test for Category Two products (topically used products made with aqueous bases or vehicles including those applied to mucous membranes) requires, for bacteria, not less than 2.0 log reduction from the initial count at 14 days, and no increase from the count at 14 days to the count at 28 days. For yeast and mold, the requirement is that there is no increase from the initial calculated count at 7, 14 and 28 days.

TABLE 1
Results of Antimicrobial Effectiveness Testing

| | | | | Log | |
|---|---|---|---|---|---|
| | | Colony | | Reduction from | |
| | Initial | Forming units | | Initial Inoculum | |
| Microorganism | Inoculum/gm | 14 Days | 28 Days | 14 Days | 28 Days |
| A. niger | 5.1 × 10⁵ | 2.0 × 10⁵ | 2.1 × 10³ | 0.4 | 2.4 |
| C. albicans | 1.6 × 10⁵ | <10 | <10 | 4.2 | 4.2 |
| E. coli | 1.1 × 10⁶ | <10 | <10 | 5.0 | 5.0 |
| P. aeruginosa | 6.0 × 10⁵ | <100 | <100 | 3.8 | 3.8 |
| S. aureus | 9.3 × 10⁵ | <10 | <10 | 5.0 | 5.0 |

From the results, the personal lubricant that includes xylitol and excludes glycerin and parabens as exemplified in Example 1 meets the current USP Category 2 criteria of acceptance for the antimicrobial preservative effectiveness test.

1. A personal lubricant composition, comprising at least 1% xylitol, wherein the formulation is free of preservatives and glycerin.

2. The personal lubricant composition of claim 1, wherein the formulation excludes parabens and glycerin.

3. The personal lubricant composition of claim 1, wherein the composition comprises from about 1% to about 15% xylitol.

4. The personal lubricant composition of claim 1, wherein the composition comprises from about 5% to about 10% xylitol.

5. The personal lubricant composition of claim 1, further comprising butylene glycol.

6. The personal lubricant composition of claim 5, wherein the composition comprises from about 10% to about 35% butylene glycol.

7. The personal lubricant composition of claim 5, wherein the composition comprises from about 15% to about 25% butylene glycol.

8. The personal lubricant composition of claim 1, further comprising propylene glycol.

9. The personal lubricant composition of claim 8, wherein the composition comprises from about 5% to about 20% propylene glycol.

10. The personal lubricant composition of claim 8, wherein the composition comprises from about 8% to about 15% butylene glycol.

11. The personal lubricant composition of claim 1, further comprising polyquaternium.

12. The personal lubricant composition of claim 11, wherein the composition comprises from about 0.1% to about 1.0% polyquaternium.

13. The personal lubricant composition of claim 11, wherein the polyquaternium is selected from the group consisting of polyquaternium-1, polyquaternium-2, polyquaternium-4, polyquaternium-5, polyquaternium-6, polyquaternium-7, polyquaternium-8, polyquaternium-9, polyquaternium- 10, polyquaternium- 11, polyquaternium- 12, polyquaternium- 13, polyquaternium- 14, polyquaternium- 15, polyquaternium- 16, polyquaternium- 17, polyquaternium- 18, polyquaternium- 19, polyquaternium-20, polyquaternium-22, polyquaternium-24, polyquaternium-27, polyquaternium-28, polyquaternium-29, polyquaternium-30, polyquaternium-31, polyquaternium-32, polyquaternium-33, polyquaternium-34, polyquaternium-35, polyquaternium-36, polyquaternium-37, polyquaternium-39, polyquaternium-42, polyquaternium-43, polyquaternium-44, polyquaternium-45, polyquaternium-46, and polyquaternium-47 and mixtures thereof.

14. The composition of claim 1, wherein the composition is clear and transparent.

15. A composition for lubricating skin or mucous membranes, comprising:
from about 1% to about 15% by weight xylitol;
from about 10% to about 35% by weight butylene glycol;
from about 5% to about 20% by weight propylene glycol;
from about 0.1% to about 1.0% by weight polyquaternium; and
from about 50% to about 70% by weight water.

16. A method of making an aqueous personal lubricant composition, comprising:
a) preparing an aqueous solution of xylitol;
b) in a container, mixing a polyquaternium with water until completely dissolved; and
c) to the polyquaternium solution, adding with mixing the xylitol solution, a butylene glycol and a propylene glycol.

17. A method of applying the personal lubricant composition of claim 1 onto the skin or a mucous membrane of a subject, comprising:
dispensing about 0.1 mL to about 10 mL of the personal lubricant composition of claim 1 onto the skin or a mucous membrane; and
rubbing the lubricant to produce a lubricating effect.

18. An article of manufacture, comprising:
a packaging material;
within the packaging material a personal lubricant composition of claim 1; and
a label that indicates that the personal lubricant composition of claim 1 is used for application to skin or a mucous membrane to provide lubricity.

19. An article of manufacture, comprising:
a personal lubricant composition of claim 1; and
a container for dispensing the personal lubricant composition.

20. A kit, comprising:
a composition of claim 1 in a package or other enclosure; and
an applicator.
